(19) United States
(12) Plant Patent Application Publication
(10) Pub. No.: US 2005/0229279 P1
(43) Pub. Date: Oct. 13, 2005

(54) Strawberry plant named MNUS 138

(76) Inventors: James J. Luby, St. Paul, MN (US); David K. Wildung, Grand Rapids, MN (US); Gene J. Galletta, Laurel, MD (US); John L. Maas, Huntington, MD (US); John M. Enns, Hyattsville, MD (US); Nada Galletta, Raleigh, NC (US)

Correspondence Address: BIOLOGICAL PATENT SERVICES, LLC, 8424 40th AVE N., NEW HOPE, MN, 55427, US

(21) Appl. No.: 11/099,728

(22) Filed: Apr. 6, 2005

Related U.S. Application Data
(60) Provisional application No. 60/560,508, filed on Apr. 8, 2004.

(51) Int. Cl. A01H 5/00

(52) U.S. Cl. PLT/208

(57) ABSTRACT

A new and distinct cultivar of Junebearing (short day) strawberry plant MNUS 138 combining the characteristics of early season ripening, high yield, excellent survival in cold temperatures, resistance to powdery mildew and red steel root rot and moderate resistance to fungal leaf spot and leaf scorch. MNUS 138 yields strawberries characterized by moderately firm flesh, a glossy appearance, moderately tough skin, and a pleasing taste with suitable use in pick-your-own production and home gardens.

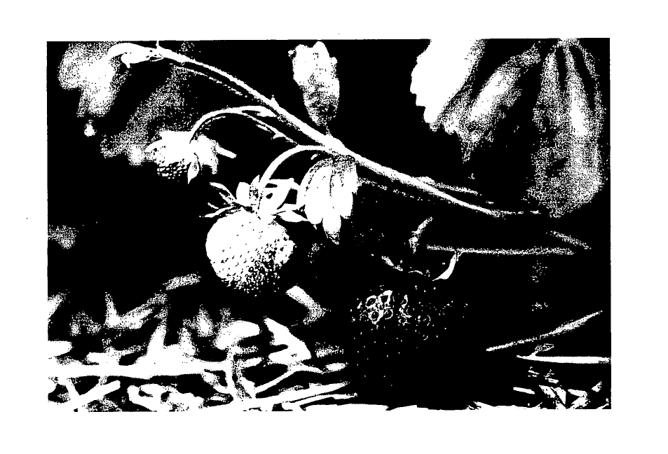

### REFERENCE TO A PROVISIONAL APPLICATION

This application claims the benefit of U.S. Provisional Application No. 60/560,508, filed Apr. 8, 2004 under U.S.C. 119(e).

### STATEMENT REGARDING FEDERALLY SPONSORED RESEARCH

Research for the development of MNUS 138 was partially funded by USDA-ARS Memorandum of Understanding No. USDA/58/1275-8M-018.

### BOTANICAL CLASSIFICATION

Fragaria ananassa

### VARIETY DENOMINATION

MNUS 138

### BACKGROUND OF THE INVENTION

MNUS 138 is a new strawberry cultivar that was raised as a seedling from a controlled cross made between strawberry cultivars Seneca (U.S. Plant Pat. No. 8,991)×Allstar (not patented) in 1983 in Beltsville, Md. MNUS 138 was selected at the University of Minnesota North Central Research and Outreach Center in Grand Rapids, Minn. in 1985. MNUS 138 was asexually propagated by stolons and planted for trials conducted from 1987 to 1990 at the University of Minnesota Horticultural Research Center near Excelsior, Minn. and the North Central Research and Outreach Center in Grand Rapids, Minn. It was further propagated by stolons and planted for evaluation in yield trials that took place from 1997 through 2003 at the Horticultural Research Center, the North Central Research and Outreach Center, the West Central Experiment Research and Outreach Center in Morris, Minn., Michigan State University in East Lansing, Mich., Pennsylvania State University in State College, Pa. and Iowa State University in Ames, Ia. Asexual reproduction of the new cultivar by stolons has shown that the unique characteristics of MNUS138 are stable and reproduced true to type in successive generations.

### BRIEF SUMMARY OF THE INVENTION

MNUS 138 is a short-day strawberry cultivar bearing fruit that matures in the early portion of the fruiting season in the Midwest United States. It is primarily adapted for growing in colder areas of the Midwest and Northeast United States. MNUS 138 is distinct from its antecedents in having an earlier period of fruiting. In trials in East Lansing, Michigan in 2000 and 2001, the mean first harvest date for MNUS 138 was 10 days before Allstar and 6 days before Seneca. The mean date on which 50% of the total yield for the season was harvested for MNUS 138 was 6 days before Allstar and 3 days before Seneca.

Of the strawberry cultivars currently grown in colder areas of the Midwest and Northeast United States, MNUS 138 is most similar to the other relatively cold hardy, early season cultivars Annapolis (not patented), Honeoye (not patented), and Sable (U.S. Patent Pending, Published Application No. 20030046739). MNUS 138 has been distinguished from these other cultivars based on the following characteristics determined in tests at the trial locations in Grand Rapids and Excelsior, Minn.:

Fruit firmness: MNUS 138 exhibits firmer fruit than Sable and Honeoye and similar fruit firmness to Annapolis.

Fruit color: When fully ripened, the skin color of MNUS 138 is lighter than that of Honeoye and more similar to fruit of Annapolis and Sable.

Average fruit weight: MNUS 138 has larger mean fruit weight than Sable and similar fruit weight to Honeoye and Annapolis.

Pubescence on the peduncle: MNUS 138 has divaricate pubescence on the peduncle whereas Annapolis and Sable have appressed pubescence.

Shape of the base of the terminal leaflet: MNUS 138 has a terminal leaflet base that is more acute than Annapolis or Sable.

Performance of MNUS 138 for yield and average berry weight is comparable to the other early season cultivars Annapolis, Sable, and Honeoye as exhibited in Table 1.

TABLE 1
Yield and mean berry weight of MNUS 138 and early cultivars
Annapolis, Sable, and Honeoye in trials at Grand Rapids, Minn.,
Morris, Minn., Excelsior, Minn., East Lansing, Mich., Ames, Iowa,
and State College, Penn.

| | Grand | | | | | |
|---|---|---|---|---|---|---|
| | Rapids | | Morris | | Excelsior | |
| | 2000 | 2001 | 2000 | 2001 | 2000 | 2001 |
| | Yield (1000 lb/acre) | | | | | |
| MNUS 138 | 13.8 | 9.0 | 7.1 | 12.2 | 3.9 | 1.4 |
| Annapolis | 6.9 | 4.4 | 0.8 | 5.9 | 4.0 | 1.5 |
| Sable | 9.3 | 10.8 | 0.7 | 11.5 | 5.1 | 5.3 |
| Honeoye | 7.7 | 9.9 | 5.1 | 14.1 | 3.9 | 7.1 |
| LSD (5%) | 6.2 | 8.8 | 4.4 | 6.2 | 2.1 | 2.5 |
| | Berry weight (g) | | | | | |
| MNUS 138 | 14.0 | 11.9 | 10.4 | 12.4 | 11.5 | 11.2 |
| Annapolis | 12.4 | 12.8 | 5.8 | 10.7 | 12.2 | 7.6 |
| Sable | 10.5 | 8.5 | 7.4 | 8.4 | 10.0 | 9.1 |
| Honeoye | 12.0 | 11.9 | 10.0 | 11.0 | 13.4 | 9.8 |
| LSD (5%) | 3.0 | 4.0 | 2.3 | 2.3 | 1.8 | 2.1 |

| | | | | | State |
|---|---|---|---|---|---|
| | East Lansing | | Ames | | College |
| | 2000 | 2001 | 2000 | 2001 | 2003 |
| | Yield (1000 lb/acre) | | | | |
| MNUS 138 | 15.1 | 2.8 | 48.3 | 23.9 | 19.6 |
| Annapolis | 9.3 | 3.7 | 9.6 | 30.1 | NA |
| Sable | 11.3 | 6.7 | 15.0 | 25.9 | 16.4 |
| Honeoye | 19.2 | 6.3 | 21.3 | 28.0 | 20.8 |
| LSD (5%) | 4.1 | 6.0 | 11.9 | 9.6 | 6.5 |
| | Berry weight (g) | | | | |
| MNUS 138 | 14.9 | 9.8 | 13.3 | 12.7 | 12.1 |
| Annapolis | 11.5 | 9.7 | 11.3 | 11.3 | NA |
| Sable | 10.5 | 10.6 | 8.9 | 9.2 | 11.2 |
| Honeoye | 13.2 | 9.1 | 13.6 | 13.1 | 11.4 |
| LSD (5%) | 1.1 | 1.3 | 5.6 | 3.7 | 1.0 |

MNUS 138 is moderately resistant to fungal leaf spot (Mycosphaerella fragariae Tul.); comparable to Annapolis and Sable and more resistant than Honeoye. MNUS 138 exhibits resistance to powdery mildew (Sphaerotheca macularis Walls ex Fr.); similar to Honeoye and more resistant than Annapolis and Sable. MNUS 138 is moderately resistant to leaf scorch (Diplocarpon earliana Ell. and Ev.) and similar to Annapolis, Honeoye, and Sable. The original plant of MNUS 138 was resistant to red stele root rot when screened with a five- race composite of Phytophthora fragariae C. J. Hickman var.fragariae as a young seedling in the greenhouse in Beltsville, Maryland in the winter of 1983-1984.

The berries of MNUS 138 have an attractive interior and exterior color and gloss. They are moderately firm, have a moderately tough skin, and have a pleasing flavor that should make the variety useful for commercial pick-your-own strawberry production and home gardens.

### BRIEF DESCRIPTION OF THE DRAWING

The accompanying color photograph shows typical fruit and a stolon tip of MNUS 138 grown under standard field conditions at Grand Rapids, Minn. The photograph depicts color features as true as is reasonably possible. The color values cited in the detailed botanical description accurately describe the new strawberry.

### DETAILED BOTANICAL DESCRIPTION

The botanical data describing MNUS138 was collected at the University of Minnesota Horticultural Research Center in Excelsior, Minn. from plants grown under standard field conditions. The characteristics may vary in detail depending on variations in conditions such as temperature, day-length, light intensity, soil types, and water and fertility levels as MNUS138 was not tested under all possible environmental conditions. The color determination is in accordance with the 1995 RHS Colour Chart of The Royal Horticultural Society, London, England, except where general color terms of ordinary dictionary significance are used.

Inflorescence:

| | |
|---|---|
| Flower position relative | Inflorescence beneath to level with |
| to foliage | foliage at bloom |
| Diameter of calyx | calyx is smaller |
| relative to corolla on | |
| secondary flowers | |
| Diameter of inner calyx | equal or slightly larger |
| relative to outer on | |
| secondary flowers | |
| Spacing of petals | usually not touching |
| Petal color (upper | 155B |
| surface) | |
| Sex type | perfect |

Size of secondary flowers

| | Mean | St. dev. | Range |
|---|---|---|---|
| Calyx diam (mm) | 19.8 | 2.3 | (15-22) |
| Corolla diam (mm) | 27.0 | 4.1 | (23-34) |
| Petal number | 5.1 | 0.4 | (5-6) |
| Petal length (mm) | 10.6 | 1.1 | (9-12) |
| Petal width (mm) | 10.6 | 1.2 | (9-12) |
| Petal length/width ratio | 1.00 | 0.05 | (0.90-1.11) |

Fruiting Truss characteristics: Attitude at first picking prostrate Peduncle pubescence moderately dense, divaricate

Fruit characteristics (data from secondary fruit except where noted):

| | | | |
|---|---|---|---|
| Fruit length (mm) | 26.1 | 1.9 | (23-29) |
| Fruit width (mm) | 31.7 | 3.4 | (29-37) |
| Fruit length/width ratio | 0.83 | 0.054 | (0.72-0.87) |

Predominant shape ovoid Difference in shape — primary more wedge-shaped and secondary more primary to secondary conic-shaped Width of band without achenes on neck of berry Medium (11-12 mm) Surface texture slightly uneven insertion of calyx calyx slightly raised Pose of calyx segments reflexed Size of calyx relative to fruit diam calyx similar or slightly smaller than fruit Adherence of calyx strong Glossiness medium External color lighter areas 45A and darker areas more exposed to sunlight 46A Internal color 45A in darker areas distal to core and 42A and 42B in lighter areas near core of receptacle Evenness of color — skin even Evenness of color — flesh lighter near core Achene coloration yellow green or red where exposed to sunlight Insertion of achenes slightly inserted Seed color on ripe fruit 151 C Seed length (mm) 1.2 Seed width (mm) 0.5
Plant characteristics: Plant habit flat-globose Bearing habit Junebearing (short day) Season of harvest early Hardiness USDA Zone 3b to 5 based on testing in Minnesota, Iowa, and Michigan.
Stolon characteristics: Anthocyanin coloration present Pubescence moderately dense, divaricate

Leaf characteristics:

| | Mean | St. dev. | Range |
|---|---|---|---|
| Terminal leaflet length | 43.8 | 3.4 | (40-48) |
| (mm) | | | |
| Terminal leaflet width | 34.7 | 2.7 | (31-38) |
| (mm) | | | |
| Terminal leaflet | 1.3 | 0.1 | (1.05-1.37) |
| length/width ratio | | | |
| Number of teeth on | 19.3 | 2.6 | (17-24) |
| terminal leaflet | | | |
| Petiole length (mm) | 78.3 | 14.8 | (62-96) |
| Stipule length (mm) | 18.5 | 2.3 | (15-21) |
| Stipule width (mm) | 8.2 | 1.2 | (6-9) |

Terminal leaflet margin profile flat to revolute Terminal leaflet shape of base mostly acute, some obtuse Terminal leaflet shape of teeth obtuse to slightly rounded Color of upper side 137A Color of under side 147B Petiole color 146C Bract frequency not present Stipule anthocyanin coloration present Interveinal blistering medium Glossiness medium Number leaflets 3 on all leaves observed Stipule pubescence present — sparse Petiole pubescence present — dense Petiole pose of hairs divaricate Angle terminal leaflet subtends to petiole 30 degrees Pubescence present on upper side of leaflet yes.

1. A new and distinct cultivar of Strawberry plant named ‘MNUS 138’ as herein illustrated and described.
